(19) United States
(12) Patent Application Publication
(10) Pub. No.: US 2026/0034051 A1
(43) Pub. Date: Feb. 5, 2026

(54) STABLE INJECTABLE CANNABIDIOL FORMULATIONS

(71) Applicant: CARDIOL THERAPEUTICS INC., Oakville (CA)

(72) Inventor: Blagoja RISTEVSKI, Oakville (CA)

(73) Assignee: CARDIOL THERAPEUTICS INC., Oakville, ON (CA)

(21) Appl. No.: 19/099,948

(22) PCT Filed: Jul. 25, 2023
(86) PCT No.: PCT/CA2023/050996

§ 371 Date: Jan. 30, 2025

Related U.S. Application Data
(60) Provisional application No. 63/397,237, filed on Aug. 11, 2022.

(51) Int. Cl. A61K 9/00; A61K 9/08; A61K 31/00; A61K 47/10; A61K 47/14; A61K 47/22

(52) U.S. Cl. CPC A61K 9/0019; A61K 9/08; A61K 31/658; A61K 47/10; A61K 47/14; A61K 47/22

(57) ABSTRACT

An injectable cannabidiol formulation for non-IV parenteral administration comprising, consisting essentially of, or consisting of: (a) a solvent system consisting of (i) an effective amount of at least one triglyceride oil; and (ii) an effective amount of at least one C₁-C₃ alkyl ester of a C₆-C₂₂ fatty acid; and (b) an effective amount of cannabidiol; wherein the formulation is free of water and ethanol, has a viscosity less than about 35 cP, and is phase stable and chemically stable. The formulation can further comprise at least one additional pharmaceutically acceptable excipient. Kits containing the formulation are also described.

### CROSS-REFERENCE TO RELATED APPLICATIONS

This application claims priority from U.S. provisional application 63/397,237 filed Aug. 11, 2022, the entire contents of which is hereby incorporated by reference.

### FIELD

The present invention relates generally to pharmaceutical formulations containing cannabinoids and, more specifically, to injectable pharmaceutical formulations containing cannabidiol.

### BACKGROUND TO THE INVENTION

Cannabinoids include phytocannabinoids which are compounds found naturally in the plant Cannabis sativa. Synthetic cannabinoids also exist and can be made by known methods. The major cannabinoid constituents of Cannabis sativa include cannabidiol and tetrahydrocannabinol (THC). THC is known to increase pulse rate, cause conjunctival reddening, and provide psychotropic (mood altering) effects such as a feeling of euphoria. In contrast, cannabidiol is non-intoxicating, and this feature makes it desirable for use as a medicinal or pharmaceutical ingredient. Its known uses include treating epilepsy, chronic pain, anxiety, insomnia, and inflammation.

Cannabinoids, including cannabidiol, are sensitive to deactivation via first-pass metabolism when taken orally. This first-pass metabolism results in low active blood levels and an overall bioavailability of less than 10%. Parenteral administration of cannabinoids can avoid this first-pass metabolism and is expected to increase bioavailability. Examples of parenteral administration include injection into a vein (intravenous, IV), into a muscle (intramuscular, IM), into the space around the spinal cord (intrathecal, IT), into the body cavity referred to as the peritoneum (intraperitoneal, IP) and beneath the skin (subcutaneous, SC).

Cannabidiol is known to be unstable in the presence of oxygen and moisture and a problem exists in formulating stable cannabidiol formulations due to the degradation of cannabidiol into other compounds, e.g., THC. Another problem relates to the insolubility of cannabidiol in water. Therefore, desirable attributes of parenteral formulations containing cannabidiol include cannabidiol stability, meaning that the cannabidiol does not break down or convert into other compounds (such as THC) to maintain efficacy and function over time; and phase stability, wherein ingredients are fully solubilized in a solvent or solvent system to provide consistent dosing and results. Other desirable attributes include a sufficiently high concentration of cannabidiol to achieve an injection volume that is sufficiently low to be practical and minimize patient discomfort; safe and non-toxic during repeated and prolonged administration when treating chronic conditions; and a sufficiently low viscosity to provide ease of administration.

Parenteral formulations containing cannabinoids are described in Rosenkrantz H, Thompson G R, Braude M C. Oral and parenteral formulations of marijuana constituents. J Pharm Sci. 1972 July; 61(7):1106-12. doi: 10.1002/jps.2600610715. PMID: 4625586; US 2007/0060638 to Olmstead et al.; WO 2008/019146A2 to Kottayil et al.; WO 2008/144475 to McAllister et al.; US 2013/0209483 to McAllister; WO 2016/147186 to Sinai; US 2019/0314296A1 to Wright et al.; WO 2019/094625A1 to Wasyl et al.; US20210393784A1 to Kingsley et al.; and WO2019140325A1 to Witowski. Nonetheless, a need still exists to provide new injectable cannabidiol formulations to meet market demands and that has one or more of the above desirable attributes.

### SUMMARY OF THE INVENTION

The inventor has found that certain alkyl esters of fatty acids are effective, in combination with a triglyceride oil, to solubilize and stabilize cannabidiol, and provide a formulation that has a low enough viscosity and high enough cannabidiol concentration to provide a cannabidiol formulation suitable for parenteral administration, other than by IV injection.

According to a first aspect, the invention provides an injectable cannabidiol formulation for non-IV parenteral administration comprising, consisting essentially of, or consisting of (a) a solvent system comprising, consisting essentially of, or consisting of (i) an effective amount of at least one triglyceride oil; and (ii) an effective amount of at least one C₁-C₃ alkyl ester of a C₆-C₂₂ fatty acid; and (b) an effective amount of cannabidiol; wherein the formulation is free of water and ethanol, has a viscosity less than about 35 cP, and is phase stable and chemically stable.

The at least one C₁-C₃ alkyl ester of a C₆-C₂₂ fatty acid can be selected from the group consisting of methyl octanoate, ethyl octanoate, isopropyl myristate, ethyl caprate, methyl oleate, and ethyl oleate. In some embodiments, the C₁-C₃ alkyl ester of C₆-C₂₂ fatty acid is selected from the group consisting of methyl octanoate and ethyl octanoate.

The at least one triglyceride oil can be at least one medium chain triglyceride. In some embodiments, the at least one medium chain triglyceride comprises esters of fatty acids in the following amounts (i) 0 to 2% w/w ester of C6 fatty acid, 50 to 65% w/w ester of C8 fatty acid, 30 to 45% w/w ester of C10 fatty acid, <2% w/w ester of C12 fatty acid, <1% w/w ester of C14 fatty acid, and <1% w/w ester of C16 or longer chain fatty acids, available commercially under the trade name Miglyol 812N.

The amount of the at least one triglyceride oil can be at least about 10% w/v, and/or can be up to about 50% w/v. Intermediate lower and upper limits are also contemplated, such as from at least about 12, 14, 16, 18, 20, 22, 24, 26, or 28% w/v, and/or up to about 48, 46, 44, 42, 40, 38, 36, 34, 32, or 30% w/v.

The amount of the at least one C₁-C₃ alkyl ester of a C6 to C22 fatty acid can be at least about 25% w/v and/or up to about 70% w/v. Intermediate lower and upper limits are also contemplated, such as from at least about 27, 29, 31, 33, 35, 37, 39, 41, 43, or 45% w/v, and/or up to about 69, 67, 65, 63, 61, 59, 57, 55, 53, 51, 49, or 47% w/v.

The ratio of the (i) at least one triglyceride oil to the (ii) at least one C₁ to C₃ alkyl ester of a C6 to C22 fatty acid can be from about 2:1 to about 1:7, or from about 1:1 to about 1:7, or from about 2:1 to about 1:6, or from about 1:1 to about 1:6. Intermediate ratios are also expressly contemplated, e.g. about 50:309, about 2000:1591, about 10:63, etc.

The cannabidiol can have a purity of at least 98.5, 99, 99.5, or 99.8%, with higher purity levels being preferred. Furthermore, the cannabidiol can be of natural origin or manufactured synthetically. A combination of natural and synthetic cannabidiol is also expressly contemplated.

The cannabidiol can be present in an amount of from about 100 mg/mL. The cannabidiol can also be present in an amount up to about 150 mg/mL, 200 mg/ml, 250 mg/mL, 300 mg/mL, or 350 mg/mL, with higher concentrations of cannabidiol being preferred to reduce injection volume and promote ease of administration while reducing patient discomfort. The identity of ingredients and their amounts in the solvent system will determine the actual amount of cannabidiol that can be fully dissolved. The inventors have found that cannabidiol can be dissolved in methyl octanoate and/or ethyl octanoate in an amount of at least 750 mg/mL. Therefore, the higher the amount of alkyl ester of fatty acid that is present, the higher the amount of cannabidiol that can be dissolved.

The formulation can have a viscosity of less than about 30 cP, about 25 cP, or about 20 cP. In general, lowering the viscosity increases ease of administration.

The formulation can further comprise, consist essentially of, or consist of an effective amount of at least one additional lipophilic pharmaceutically acceptable excipient, which can be selected from the group consisting of preservatives, antioxidants, anesthetic compounds, viscosity modifying agents, and pharmacokinetic modifying agents.

Example lipophilic antioxidants are those selected from the group consisting of propyl gallate, Vitamin E, ascorbyl palmitate, butylated hydroxytoluene (BHT) and butylated hydroxyanisole (BHA). These compounds and other lipophilic antioxidants and preservatives are used to improve shelf-life. In some embodiments, the at least one lipophilic antioxidant is Vitamin E and/or ascorbyl palmitate. The total amount of these antioxidants can be from about 0.01, 0.05, or 0.1% w/v and up to about 1.5, 1.0, 0.8, 0.7, 0.6, or 0.5% w/v.

An example lipophilic anesthetic compound is benzyl alcohol which can be present in an amount from about 0.5% w/v to about 1.5% w/v. Benzyl alcohol also functions as an additional lipophilic solvent.

In some embodiments, the formulation is free of phosphatidylglycerols and/or salts thereof. In the same or other embodiments, the formulation is free of POPG-Na (2-Oleoyl-1-palmitoyl-sn-glycero-3-phospho-rac-(1-glycerol) sodium salt). In the same or other embodiments, the formulation is free of fatty acids, such as C₆-C₂₂ fatty acids. In some embodiments, the formulation is free of octanoic acid.

In the same or other embodiments, the present formulation is free of other surfactants, polar lipids, and/or additional solvents. These include surfactants selected from the group consisting of polyoxyethylene (20) sorbitan monooleate (also called polysorbate 80 and Tween 80); macrogol hydroxystearate (which is a mixture of mainly mono esters and diesters of 12-hydroxystearic acid and macrogols obtained by the ethoxylation of 12-hydroxystearic acid (the number of moles of ethylene oxide reacted per mole of 12-hydroxystearic acid is 15 and proprietary versions include Solutol™ HS15, Crodasol™ HS and Kolliphor™ HS 15); poloxamers (which are triblock co-polymers of a central hydrophobic chain of polyoxypropylene flanked by two hydrophilic chains of polyoxyethylene; proprietary brands include Pluronics™); and glycocholate. Polar lipids that can be excluded include glycerophospholipids (GPLs) such as phospholipids, glycolipids, sphingolipids, glycerol monooleate and glycerol monostearate. Solvents that can be excluded include other aliphatic alcohols (e.g. methanol, propanol, etc.), C₈-C₂₂ cyclic alcohols, aromatic alcohols, sesame oil, amphiphilic liquid polymeric solvents such as propylene glycol, polyethylene glycols (e.g. PEG-100, PEG-200, PEG-300, PEG-400, PEG-600, PEG-800, PEG-1000, and PEG-2000), polyethoxylated glycerides, polyethylene glycol esters, N-(2-hydroxypropyl)methacrylamide (HPMA), and polyvinylpyrrolidone (PVP) (e.g. K12 or K17). In the same or other embodiments, the formulation is free of 2, 3, 4, or 5 of the above compounds or ingredients.

The formulations are sterile and can be sterilised by methods known in the art. They can be administered via subcutaneous (SC), intramuscular (IM), intraperitoneal (IP), and/or intrathecal (IT) injection and be provided in a variety of formats including as part of a kit. Thus, according to a second aspect, the invention provides a kit comprising (i) a container which comprises the formulation according to the first aspect; and (ii) instructions for use of the formulation. The container can be part of a single-dose or multi-dose injector, vial, cartridge, or pre-filled syringe.

### DETAILED DESCRIPTION

### Definitions

For the sake of clarity and to avoid ambiguity, certain terms are defined herein as follows.

The term “solvent system” is used to refer to a mixture of ingredients which, together, function to solubilize cannabidiol.

“Single-phase” means that the solution is homogeneous and that no solids and distinct liquid phases are present.

The term “room temperature” is used interchangeably with “ambient temperature” and means a temperature of between 18° C. and 25° C.

As used herein the term “pharmaceutically active agent” means a drug or agent which can be employed as disclosed herein and is intended to be used in a subject to treat or prevent diseases, ailments, physical damage, or pathological symptoms; or to influence the state, the condition or the functions of the body or mental states. Drugs in use can be found in reference works such as, for example, the Rote Liste
or the Merck Index. Examples which may be mentioned include, for example, cannabidiol.

When a composition of matter is described as having a “purity of X %” this means that one or more impurities may be present in an amount up to 100-X % by weight, based on the total weight of the composition of matter. The purity of an ingredient can be determined by high performance liquid chromatography (HPLC) or other suitable means.

The term “subject” means members of the animal kingdom including humans and other mammals.

When used herein, the terms “treat,” “treatment,” and the like expression mean stopping or delaying the progression of a condition, disorder, or disease. The terms “prevent,” “prevention,” and the like expression mean preventing or delaying the onset of a condition, disorder, or disease. The terms are intended to encompass “improving quality of life,” “extending the life,” and “improving clinical outcomes” of a subject suffering from, or at risk of suffering from, the condition, disorder, or disease, and do not necessarily mean “curing” the condition, disorder, or disease, though such is not precluded.

“Pharmaceutically acceptable excipient” when used herein means any substance which can be formulated with or that is present alongside a pharmaceutically active agent to achieve a desired function or functions and that is not biologically or otherwise undesirable in an injectable dosage form. For example, the excipient must be non-toxic when injected and compatible with the other ingredients in the formulation. The person skilled in the art will appreciate what compounds or ingredients would qualify as a pharmaceutically acceptable excipient given the teachings of the present specification and information in the public domain.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The terms “a” or “an” means “one or more.” As such, the terms “a” (or “an”), “one or more” and “at least one” can be used interchangeably herein. It should also be noted that the term “or” is generally employed in the sense of “and/or” unless the context clearly dictates otherwise.

The term “comprising” means “including without limitation.” Thus, a formulation comprising a list of ingredients may include additional ingredients not expressly recited. It is also to be noted that the terms “comprising,” “including,” “containing,” and “having” can be used interchangeably.

The term “consisting of” means including the listed ingredients and such additional ingredients as may be present in the listed ingredients as natural or commercial impurities or additives. Natural and commercial impurities and additives will be apparent to the person of ordinary skill in the art. For example, synthetic cannabidiol may contain up to about 0.5% w/w of impurities such as residual solvents and by-products of the manufacturing process.

Formulations according to the invention are phase stable. When used herein, “phase stable” or the like expression means that the formulation forms a clear, single-phase solution both upon preparation and after storage in an air-tight sealed container at a temperature ranging from about 2° C. to about 25° C. for a period of at least 12 months. The present formulations are also chemically stable which means that the cannabidiol does not substantially convert to THC during storage under the same conditions and time frame. Conversion to up to 10 ppm THC based on the total formulation is considered not to compromise the chemical stability.

The term “consisting essentially of” means “including the listed ingredients and any additional ingredients that do not materially affect the basic and novel properties of the invention.” By “basic and novel properties” is meant the phase stability of the present formulations as defined above, and the suitability of the formulation as a non-IV parenteral dosage form (e.g., SC injectable, IM injectable, IP injectable, IT injectable, etc.), for the treatment or prevention of a condition, disease, or disorder.

The terms “% w/w,” “% wt.,” “w/w %,” “wt. %” and variations thereof, mean the amount of a substance in terms of the weight of that substance divided by the total weight of the formulation containing that substance, and multiplied by 100.

The terms “% w/v,” “w/v %” and variations thereof, mean the mass of a solute in grams divided by the volume of the solution in which the solute is dissolved in mL, and multiplied by 100. For example, a formulation containing 10 grams of a solvent per 25 mL of the solution that contains the solvent, would have 40 w/v % of the solvent calculated as follows: (10÷25)×100=40 w/v %.

The term “about” refers to variations in an expressed numerical quantity that can occur, for example, through measuring and liquid handling procedures used for making pharmaceutical formulations, differences in the manufacture, source, or purity of the ingredients used to make the formulations, and/or differences due to different equilibrium conditions or different reaction levels of ingredients in a formulation resulting from an initial mixture. For the sake of clarity, the term “about” includes variations in the expressed value up to ±5% or up to +10%. Whether or not a value is modified by the term “about,” the claims include equivalents to the values.

When used herein, the term “effective amount” means an amount that would bring about the desired effect, based on the purpose and function of the ingredient in the context of the invention disclosed herein. Implicit in “desired effect” is that toxicity does not arise. For example, an effective amount of a pharmaceutically active agent is that amount which would be effective to provide a therapeutic effect while avoiding toxicity, such as may occur via long-term administration. An effective amount of a solvent is that amount which, alone or together with other ingredients, would be effective to solubilize the other or remaining ingredients of the formulation. What constitutes an effective amount will be determinable by the person of ordinary skill in the art by routine experimentation, having regard to the teachings herein.

When used herein, the expression “free of Y” means that “Y” is not deliberately added but may be present as an impurity or due to other factors. For example, in the case of the present formulations that are free of water, the formulation may contain minute amounts of water due to the formulation or ingredients thereof being exposed to water in the atmosphere or containing water as an impurity. For the sake of clarity, a formulation that is “free of Y” will not contain Y or contain only up to 0.01, 0.02, 0.03, 0.04, 0.05, 0.06, 0.07, 0.08, 0.09, 0.1, 0.2, 0.3, 0.4, or 0.5% w/v, based on the total formulation.

The values recited herein are intended to include all values that meet the stated parameters including those not expressly recited. For example, a value of less than 1.0 is intended to include less than 0.99, less than 0.98, less than 0.97, less than 0.90, less than 0.84, less than 0.56, less than 0.01, etc. Thus, all ranges disclosed herein are to be understood to encompass any and all subranges subsumed therein. For example, a stated range of “1 to 10” should be considered to include any and all subranges between (and inclusive of) the minimum value of 1 and the maximum value of 10, e.g., 1 to 6.3, 5.5 to 10, 2.7 to 6.1, etc.

The present specification contemplates the possibility of omitting any ingredients even if they are not expressly named as included or excluded in the specification.

### Solvent System

The present formulations use a solvent system to dissolve at least 100 mg cannabidiol per mL of the formulation while achieving a viscosity of no more than 35 centipoise (cP). The solvent system consists of an effective amount of at least one triglyceride oil and an effective amount of at least one C₁-C₃ alkyl ester of a C₆-C₂₂ fatty acid. The greater the ratio of the C₁-C₃ alkyl ester of the C₆-C₂₂ fatty acid to the triglyceride oil, the lower the viscosity of the final formulation, and also the higher the amount of cannabidiol that can be fully dissolved.

### Triglyceride Oils

Triglyceride oils are lipid molecules with three fatty acids attached to a glycerol backbone.

Medium-chain fatty acids (MCFAs), along with more abundant long-chain fatty acids (LCFAs), are natural compounds present in both animal and plant tissues that participate in cell metabolism. MCFAs, being monocarboxylic acids with a hydrocarbon chain length of 6 to 12 total carbon atoms, are abundant in nature, although they are present in plant and animal material at much smaller quantities than LCFAs with a hydrocarbon chain length of 14 to 22 total carbon atoms. The lipophilicity of MCFAs and LCFAs, measured as partition of the free acid between water and heptane, gradually increases with increasing carbon atom chain length.

Triglyceride oils are liquid at room temperature. Similar to simple fatty acids, the length of the fatty acid group determines the nomenclature of the triglycerides, e.g., short-chain triglycerides (SCTs), medium-chain triglycerides (MCTs), and long-chain triglycerides (LCTs).

Preferably, the at least one triglyceride oil is one or more medium chain triglycerides (MCTs). “Medium chain triglyceride” refers to esters of glycerol having three C6 to C12 fatty acid chains, wherein the three fatty acid chains may be the same or different. Medium chain triglycerides are represented by the following formula:

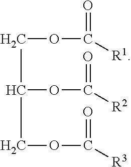

wherein each of R¹, R², and R³ independently represents a fatty acid molecule with 6 to 12 carbon atoms. A fatty acid molecule having 6 carbons is referred to as a C6 fatty acid. A fatty acid molecule having 8 carbons is referred to as a C8 fatty acid. Likewise, a fatty acid molecule having 10 carbons is referred to as a C10 fatty acid. In various embodiments, each R group is the same. In other embodiments, two are the same and one is different. In still other embodiments, all are different.

The medium chain triglyceride may be synthetic or natural (e.g., produced from SUBSTITUTE SHEET (RULE 26) fractionated oils, such as coconut oil and/or palm kernel oil) as is known in the art.

In various embodiments, the medium chain triglyceride comprises esters of (i) three C8 fatty acids; (ii) three C10 fatty acids; (iii) two C8 fatty acids and one C10 fatty acid; (iv) two C10 fatty acids and one C8 fatty acid; (v) two C8 fatty acids and one C6 fatty acid; (vi) two C10 fatty acids and one C6 fatty acid; (vii) one C8 fatty acid, one C10 fatty acid, and one C6 fatty acid; or (viii) any other combination of C6, C8, C10, and C12 fatty acids.

The skilled artisan will appreciate that a mixture of medium chain triglycerides may result from any process (e.g., fractionation, hydrogenation) used to prepare medium chain triglycerides. The MCT or MCTs may comprise one, two, three, four or more different medium chain triglycerides. For example, substantially all the medium chain triglycerides obtained from fractionated coconut oil may comprise C8 and/or C10 fatty acids; however, there may be some medium chain triglycerides containing C6 and/or C12 fatty acids. In one embodiment, the MCTs comprise two different medium chain triglycerides, where a first medium chain triglyceride comprises esters of two C8 fatty acids and one C10 fatty acid and a second medium chain triglyceride comprises esters of one C8 fatty acid and two C10 fatty acids.

In one embodiment, the medium chain triglycerides comprise esters of (i) 0 to 2% w/w C6 fatty acid, 65 to 80% w/w C8 fatty acid, 20 to 35% w/w C10 fatty acid, and 0 to 2% w/w C12 fatty acid; (ii) 0 to 2% w/w C6 fatty acid, 50 to 65% w/w C8 fatty acid, 30 to 45% w/w C10 fatty acid, and 0 to 2% w/w C12 fatty acid; or (iii) 0 to 2% w/w C6 fatty acid, 45 to 65% w/w C8 fatty acid, 30 to 45% w/w C10 fatty acid, 0 to 3% w/w C12 fatty acid, and 0 to 5% w/w linoleic acid. In one embodiment, the medium chain triglyceride comprises esters of 0 to 2% w/w C6 fatty acid, 50 to 65% w/w C8 fatty acid, 30 to 45% w/w C10 fatty acid, 0 to 2% w/w C12 fatty acid, <1% w/w C14 fatty acid, and <1% w/w C16 or longer chain fatty acids, and which is commercially available as MIGLYOL® 812N. The weight % values recited in this paragraph are based on the weight of the total fatty acid content of the triglycerides. Other commercial MCTs that can be used are those sold in association with the trade names MIGLYOL® 810, 818, and 829 (from supplier, IOI Oleochemical GmbH, Germany).

Preferably, the formulation comprises MCTs containing a mixture of C8 and C10 triglycerides in a ratio (C8:C10) of from about 55:45 to about 65:35.

Still other triglyceride oils that can be used are those containing omega-3 fatty acids, and omega-6 fatty acids. Omega-3 fatty acids include (C18) alpha-linolenic acid (ALA), (C20) eicosapentaenoic acid (EPA), and (C22) docosahexaenoic acid (DHA). Omega-6 fatty acids include (C18) linolenic acid (LA), and (C20) arachidonic acid (AA).

In an embodiment, the formulation is free of unmodified fatty acids such as, for example, C₆-C₂₂ fatty acids. An exemplary C6-C22 fatty acid is octanoic acid.

### C1-C3 Alkyl Esters of C6-C22 Fatty Acids

C1-C3 alkyl esters of C6-C22 fatty acids that are liquid at room temperature are used as a co-solvent in the present co-solvent systems. The inventors have found that these compounds are useful for solubilizing higher amounts of cannabidiol than triglycerides and, when incorporated into the present formulations, also serve to lower the viscosity of the final formulation while preventing the conversion of cannabidiol into other compounds such as THC. Examples of C1-C3 alkyl esters of fatty acids that can be used include methyl and ethyl esters of octanoic acid (i.e., methyl octanoate and ethyl octanoate, respectively).

Other C1-C3 alkyl esters of C6-C22 fatty acids that can be used are C1-C3 alkyl esters of omega-3 fatty acids, and omega-6 fatty acids. Omega-3 fatty acids include (C18) alpha-linolenic acid (ALA), (C20) eicosapentaenoic acid (EPA), and (C22) docosahexaenoic acid (DHA). Omega-6 fatty acids include (C18) linolenic acid (LA), and (C20) arachidonic acid (AA).

### Cannabidiol

The present formulations contain an effective amount of cannabidiol, which is the compound having the below chemical structure:

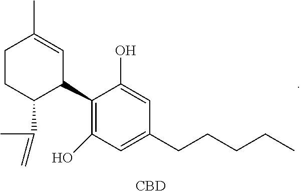

The cannabidiol can be of natural or synthetic origin. Methods of manufacturing synthetic cannabidiol are known in the art. For example, the cannabidiol from PURISYS, LLC (headquartered in Athens, Georgia, U.S.A) is made according to processes such as those described in U.S. patent publication nos. US 2017/0008868 A1 (granted as U.S. Pat. No. 10,059,683) and US20180319763A1 (granted as U.S. Pat. No. 10,844,035), incorporated herein by reference. Synthetic cannabidiol made by other processes and manufacturers can also be used to make the present formulations. Botanically sourced cannabidiol can be derived from a variety of cannabis plants, including hemp, and purified using conventional means. cannabidiol that can be used to make the present formulations can be in crystalline or oil form prior to dissolution in the solvent system. All commercial sources of cannabidiol are useful in the context of the present invention.

Preferably, the cannabidiol has a purity of at least 99.5%, 99.6%, 99.7%, 99.8% or 99.9%. Since the present formulations contain compounds/ingredients other than cannabidiol, any impurities present in the cannabidiol will be present in a much smaller amount in the overall formulation.

Impurities that may be present in synthetic cannabidiol include residual solvents (e.g., methanol, n-heptane, dichloromethane, and triethylamine) and/or by-products or residues of manufacture, e.g., olivetol, monobromo-cannabidiol, and delta-9-THC. Botanically sourced cannabidiol may contain small amounts of other cannabinoids (e.g., cannabidivarin (CBDV) and butyl analog of cannabidiol (cannabidibutol)), terpenes, and solvents or ingredients used in the purification process as well as pesticide residues.

The terms tetrahydrocannabinol, THC, delta-9-tetrahydrocannabinol, and delta-9-THC are used interchangeably herein and refer to the chemical compound having the structure shown below.

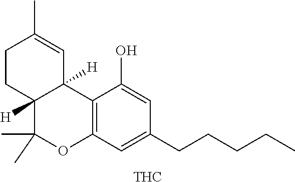

The term “THC” is used broadly herein to include the double bond isomers and their stereoisomers.

The present formulations are free of THC, meaning that THC is either not present or is present in any amount less than 0.5% w/w, preferably less than 0.4% w/w, even more preferably less than 0.3% w/w, and still more preferably less than 0.2% w/w based on the weight of the formulation. To avoid psychotropic or intoxicating effects, it would be desirable to reduce the level of THC to as low a level as possible. Embodiments of present formulations have no THC or a concentration of THC less than 10, 9, 8, 7, 6, 5, 4, 3, 2, or 1 ppm based on the total formulation.

The concentration of cannabidiol in the present formulations can be from about 100, 150, or 200 mg/mL. Additionally, or alternatively, it can be up to about 350, 300, or 250 mg/mL.

### Additional Ingredients

The present formulations can contain additional pharmaceutically acceptable excipients and pharmaceutically active agents, provided that these ingredients do not undermine the basic and novel properties of the invention.

For example, the present formulations can employ an effective amount of at least one lipophilic antioxidant to prevent oxidation and degradation of the compounds during storage. The at least one antioxidant can be selected from the group consisting of vitamin E (also referred to as α-tocopherol), carotenoids (xanthophylls and carotenes), propyl gallate, lecithin, ascorbyl palmitate, butylated hydroxyanisole (BHA), butylated hydroxytoluene (BHT), and monothioglycerol tert-butylhydroquinone (TBHQ). What constitutes an “effective amount” depends on the identity of the compound. In general, the total amount of antioxidant(s) will be more than about 0.01% w/v and less than about 1.5% w/v. In some embodiments, the amount is more than about 0.1% w/v and less than about 1% w/v.

Additional excipients which are contemplated for use in the practice of the disclosure are those available to those of ordinary skill in the art, for example, those found in the United States Pharmacopeia—National Formulary Vol. XVII, U.S. Pharmacopeia Convention, Inc., Rockville, Md. (1989).

### EXAMPLES

Specific examples will now be described wherein the following abbreviations are used.

### LIST OF ABBREVIATIONS

° C.—degrees Celsius
cP—centipoise
mL—milliliter
Pre-TS—pre-terminal sterilization procedure
QS—quantum sufficit
RH—relative humidity
T—time
TS—terminal sterilization
wk(s)—week(s)

In the following examples, the raw materials in Table A were used.

TABLE A

| | Chemical | % active | |
|---|---|---|---|
| | Name or | concentration/ | Supplier(s)/ |
| Ingredient | Trade Name | purity | CAS No. |
| Synthetic | Cannabidiol | Assay: 100% ± 2% | Purisys Inc./ |
| Cannabidiol | | | 13956-29-1 |
| MCT | Miglyol 812N | Fatty acid C6: ≤2%; | Azelis Canada/ |
| (medium | | Fatty acid C8: 50-65%; | 73398-51-5 |
| chain | | Fatty acid C10: 30-45%; | Manufacturer |
| triglycerides) | | Fatty acid C12: ≤2%; | is IOI |
| | | Fatty acid C14: ≤1%; | Oleochemical |
| | | Fatty acid ≥ C16: ≤1% | |
| POPG-Na | 2-Oleoy1-1- | ≥98.0% | Sigma-Aldrich |
| | palmitoyl-sn- | | and Lipoid |
| | glycero-3- | | GmbH/202070- |
| | phospho-rac- | | 86-8 |
| | (1-glycerol) | | |
| | sodium salt | | |
| Benzyl | Benzyl | ≥99.75% | Sigma-Aldrich/ |
| alcohol | alcohol - | | 202070-86-6 |
| | anhydrous | | |
| Methyl | Methyl | ≥99.9% | Sigma-Aldrich/ |
| Octanoate | Octanoate | | 111-11-5 |
| Ethyl | Ethyl | 99.7% | Sigma-Aldrich/ |
| Octanoate | Octanoate | | 106-32-1 |

### Example 1

Formulations 1-8 were prepared and are summarized in Table 1 below. The amounts shown are amounts of the raw materials used. Actual concentrations can be calculated with reference to Table A. All formulations were formulated to provide 250 mg cannabidiol/mL of the formulation (the “label claim”).

TABLE 1

| | 1 | 2 | 3 | 4 | 5 | 6 | 7 | 8 |
|---|---|---|---|---|---|---|---|---|
| Ingredient | % w/v | | | | | | | |
| cannabidiol | 25 | 25 | 25 | 25 | 25 | 25 | 25 | 25 |
| Miglyol | 10 | 40 | 10 | 10 | 10 | 10 | 40 | 40 |
| 812N | | | | | | | | |
| POPG-Na | 2 | 2 | — | — | — | — | — | — |
| Benzyl | 1.18 | 1.18 | 1.18 | 1.18 | — | — | 1.18 | 1.18 |
| alcohol | | | | | | | | |
| Octanoic | QS | QS | — | — | — | — | — | — |
| acid | | | | | | | | |
| Methyl | — | — | QS | — | QS | — | QS | — |
| octanoate | | | | | | | | |
| Ethyl | — | — | — | QS | — | QS | — | QS |
| octanoate | | | | | | | | |

Formulations 1 and 2 are not in accordance with the present invention as they do not contain an alkyl ester of a fatty acid, let alone a C1-C3 alkyl ester of a C6-C22 fatty acid. Instead, they contain a co-solvent (octanoic acid), and a surfactant/emulsifier POPG-Na. Formulations 3-8 contain either a C1 alkyl ester of a C8 fatty acid (methyl octanoate) or a C2 alkyl ester of a C8 fatty acid (ethyl octanoate), and no POPG-Na.

Samples of each formulation were prepared using the method described below in Example 2, filled into clear glass vials, and the glass vials were manually stoppered and sealed.

Table 2 shows the amount of cannabidiol and THC in samples of Formulations 1 to 8 at time=0, i.e., immediately following preparation.

TABLE 2

| cannabidiol Formulation | % Label Claim | d9-THC Limit |
|---|---|---|
| # | Cannabidiol | (ppm) |
| 1 | 98.4 | 17 |
| 2 | 98.6 | 3 |
| 3 | 98.5 | ND |
| 4 | 98.8 | ND |
| 5 | 98.9 | ND |
| 6 | 98.7 | ND |
| 7 | 98.4 | ND |
| 8 | 98.7 | ND |

ND = not detected

The amount of cannabidiol expressed above is expressed in terms of the % of label claim, wherein the label claim is 250 mg/mL.

The viscosity of samples of Formulations 1 to 8 was measured at room temperature, at time=0, and the results are shown in Table 3 below.

TABLE 3

| Formulation | Viscosity (cSt) |
|---|---|
| 1 | 21 |
| 2 | 42 |
| 3 | 5 |
| 4 | 6 |
| 5 | 5 |
| 6 | 6 |
| 7 | 15 |
| 8 | 17 |

Other samples of Formulations 1 to 8 were stored for two weeks at about 5° C. (between 2° C. and 8° C.) at a relative humidity of 60±5%. The cannabidiol and THC content of these other samples were measured and are shown in Table 4 below.

TABLE 4

| | % Label Claim | d9-THC Limit |
|---|---|---|
| Formulation | Cannabidiol | (ppm) |
| 1 | 99.1 | 41 |
| 2 | 98.6 | 10 |
| 3 | 99.0 | ND |
| 4 | 98.9 | ND |
| 5 | 99.7 | ND |
| 6 | 99.2 | ND |
| 7 | 99.4 | ND |
| 8 | 99.4 | ND |

Still other samples of Formulations 1 to 8 were stored at about 25° C. (about 2° C.) for two weeks at a relative humidity of 60±5%. These samples were evaluated for their cannabidiol and THC content at room temperature and the results are summarized in Table 5.

TABLE 5

| | % Label Claim | d9-THC Limit |
|---|---|---|
| Formulation | Cannabidiol | (ppm) |
| 1 | 99.0 | 235 |
| 2 | 98.8 | 58 |
| 3 | 98.7 | ND |
| 4 | 99.7 | ND |
| 5 | 99.5 | ND |
| 6 | 99.3 | ND |
| 7 | 99.2 | ND |
| 8 | 99.5 | ND |

The results shown in Tables 4 and 5 show that THC was not detected in Formulations 3 to 8 after storage for two weeks at temperatures ranging from 2° C. to 27° C. However, Formulations 1 and 2 showed THC formation both initially and after storage under the same conditions.

Other samples of Formulations 1 to 8 were stored for four weeks at either 5° C. (±3° C.) or 25° C. (±2° C.) and at a relative humidity of 60±5%. The viscosity of these other samples at time=4 weeks, measured at room temperature, is summarized in Table 6.

TABLE 6

| | Viscosity (cP) | Viscosity (cP) |
|---|---|---|
| | (after 4 wks of | (after 4 wks of |
| | storage at 5° C. | storage at 25° C. |
| Formulation | (±3° C.)) | (±2° C.)) |
| 1 | 21 | 21 |
| 2 | 42 | 41 |
| 3 | 5 | 5 |
| 4 | 6 | 6 |
| 5 | 5 | 5 |
| 6 | 6 | 6 |
| 7 | 15 | 15 |
| 8 | 17 | 17 |

Tables 3 and 6 show that the viscosity remained substantially constant over a four-week period, and that all formulations, except for Formulation 2, had a viscosity less than 35 cP and were therefore deemed suitable for injection according to the present invention. The viscosity of Formulation 2 was above 35 cP and not considered suitable for injection according to the present invention.

Formulations 1 to 8 were clear and in a single phase at the time of preparation and they remained so throughout the experiments described above.

Collectively, these results show that formulations having acceptable viscosity, chemical stability, phase stability, and concentration of cannabidiol can be formulated using the ingredients of Formulations 3-8 in the amounts specified in Table 1. They also show that chemical stability of cannabidiol is compromised when the methyl octanoate or the ethyl octanoate is replaced with their fatty acid counterpart, namely, octanoic acid, in combination with POPG-Na at 2% w/v.

### Example 2—Effect of Terminal Sterilization on Stability

An experiment was performed to determine the effect of a terminal sterilization procedure on the stability of formulations according to the invention. Formulations 9 to 12 having a label claim of 250 mg/mL cannabidiol were prepared and are summarized in Table 7.

TABLE 7

| | | | |
|---|---|---|---|
| 9 | 10 | 11 | 12 |

| | |
|---|---|
| Ingredient | % w/v |

| | | | | |
|---|---|---|---|---|
| cannabidiol | 25 | 25 | 25 | 25 |
| Miglyol 812N | 10 | 40 | 10 | 40 |
| Benzyl alcohol | 1.18 | 1.18 | 1.18 | 1.18 |
| Methyl octanoate | QS | QS | — | — |
| Ethyl octanoate | — | — | QS | QS |

As shown above, Formulations 9-12 contain the same ingredients and in the same amounts as Formulations 3, 7, 4, and 8, respectively.

A 500-mL batch of each formulation was prepared as follows:
1. A selected amount by weight of methyl octanoate or ethyl octanoate and Miglyol 812N were dispensed into a main formulation vessel.
2. A selected amount by weight of cannabidiol was added to the formulation vessel while stirring using a magnetic stir bar until completely dissolved.
3. A selected amount by weight of benzyl alcohol was added while stirring to obtain a homogenous solution.
4. The solution was then transferred into a 500 mL volumetric flask, and an amount of methyl octanoate or ethyl octanoate was added QS to 500-mL.
5. The flask was stoppered and mixed by inversion at least 20 times.
6. 2 mL aliquots of each formulation were dispensed into 3-mL Type I clear glass vials. The vials were then stoppered with grey Flurotec 4588/40 13 mm stoppers, WFI washed, and sealed with 13 mm Flip Off TruEdge™ 6-Bridge seals. A total of 135 filled, labeled, and sealed vials were prepared.
   In steps 1 to 4 above, the amounts of ingredients were selected to achieve a final concentration outlined in Table 7 above.

Bulk samples of the four (4) formulations were visually inspected for their appearance and analyzed for their cannabidiol content, delta-9 THC content, and viscosity. Vialed samples of all four (4) formulations were removed for pre-terminal sterilization (pre-TS) analytical tests. The balance of the vialed samples was subjected to autoclave sterilization with steam at 121° C. for 35 minutes (“overkill cycle”) (“terminal sterilization” or a “TS”). Different vialed samples were subjected to different conditions in a stability chamber as follows:
a. 5° C. 3° C. and 60% RH 5% RH,
b. 25° C.±2° C. and 60% RH±5% RH, or
c. 40° C. 2° C. and 75% RH 5% RH Samples were pulled at T=0, T=1 wk, T=2 wks and T=4 wks and visually inspected for their appearance and analyzed for their cannabidiol content, delta-9 THC content, and viscosity. The results are summarized in Tables 8, 9, 10, and 11.

TABLE 8
Bulk Samples

| | % Label | d9-THC | Viscosity | |
|---|---|---|---|---|
| | Claim | Limit | Data | |
| Formulation | Cannabidiol | (ppm) | (cP) | Colour |
| 9 | 99.7 | ND | 4.6 | Pale yellow |
| 10 | 100.0 | ND | 15.0 | Pale yellow |
| 11 | 100.1 | ND | 5.5 | Pale yellow |
| 12 | 99.8 | ND | 15.9 | Pale yellow |

Pre-TS; 5° C. ± 3° C.; 60% ± 5% RH

| | | | | |
|---|---|---|---|---|
| 9 | 99.6 | ND | 4.6 | Pale yellow |
| 10 | 99.7 | ND | 15.0 | Pale yellow |
| 11 | 99.4 | ND | 5.5 | Pale yellow |
| 12 | 99.7 | ND | 15.9 | Pale yellow |

Post-TS; T = 0; 5° C. ± 3° C.; 60% ± 5% RH

| | | | | |
|---|---|---|---|---|
| 9 | 99.5 | 3 | 4.6 | Yellow |
| 10 | 99.5 | 3 | 15.0 | Yellow |
| 11 | 98.4 | 3 | 5.5 | Yellow |
| 12 | 99.2 | 3 | 15.9 | Yellow |

TABLE 9

| | | | | |
|---|---|---|---|---|
| | % Label | d9-THC | Viscosity | |
| | Claim | Limit | Data | |
| Formulation | Cannabidiol | (ppm) | (cP) | Colour |

Post-TS; T = 2 wks; 5° C. ± 3° C.; 60% ± 5% RH

| | | | | |
|---|---|---|---|---|
| 9 | 102.1 | 3 | 4.6 | Pale yellow |
| 10 | 103.4 | 3 | 15.0 | Pale yellow |
| 11 | 101.1 | 3 | 5.5 | Pale yellow |
| 12 | 101.8 | 3 | 15.9 | Pale yellow |

Post-TS; T = 4 wks; 5° C. ± 3° C.; 60% ± 5% RH

| | | | | |
|---|---|---|---|---|
| 9 | 102.1 | 3 | 4.6 | Pale yellow |
| 10 | 102.7 | 3 | 15.0 | Pale yellow |
| 11 | 101.8 | 3 | 5.5 | Pale yellow |
| 12 | 102.3 | 3 | 15.9 | Pale yellow |

TABLE 10

| | | | | |
|---|---|---|---|---|
| | % Label | d9-THC | Viscosity | |
| | Claim | Limit | Data | |
| Formulation | Cannabidiol | (ppm) | (cP) | Colour |

Post-TS; T = 2 wks; 25° C. ± 2° C.; 60% ± 5% RH

| | | | | |
|---|---|---|---|---|
| 9 | 100.8 | 3 | 4.6 | Yellow |
| 10 | 102.2 | 3 | 15.0 | Yellow |
| 11 | 102.3 | 3 | 5.5 | Yellow |
| 12 | 101.8 | 3 | 15.9 | Yellow |

Post-TS; T = 4 wks; 25° C. ± 2° C.; 60% ± 5% RH

| | | | | |
|---|---|---|---|---|
| 9 | 103.0 | 3 | 4.6 | Yellow |
| 10 | 102.0 | 3 | 15.0 | Yellow |
| 11 | 102.6 | 3 | 5.5 | Yellow |
| 12 | 102.1 | 3 | 15.9 | Yellow |

TABLE 11

| | | | | |
|---|---|---|---|---|
| | % Label | d9-THC | Viscosity | |
| | Claim | Limit | Data | |
| Formulation | Cannabidiol | (ppm) | (cP) | Colour |

Post-TS T = 1 wk; 40° C. ± 2° C.; 75% ± 5% RH

| | | | | |
|---|---|---|---|---|
| 9 | 99.0 | 3 | 4.6 | Yellow |
| 10 | 99.4 | 3 | 15.0 | Yellow |
| 11 | 99.5 | 3 | 5.5 | Yellow |
| 12 | 99.4 | 3 | 15.9 | Yellow |

Post-TS T = 2 wk; 40° C. ± 2° C.; 75% ± 5% RH

| | | | | |
|---|---|---|---|---|
| 9 | 102.4 | 3 | 4.6 | Yellow |
| 10 | 100.9 | 3 | 15.0 | Yellow |
| 11 | 101.8 | 3 | 5.5 | Yellow |
| 12 | 102.7 | 3 | 15.9 | Yellow |

Post-TS T = 4 wk; 40° C. ± 2° C.; 75% ± 5% RH

| | | | | |
|---|---|---|---|---|
| 9 | 102.5 | 3 | 4.6 | Golden yellow |
| 10 | 102.6 | 4 | 15.0 | Golden yellow |
| 11 | 101.5 | 3 | 5.5 | Golden yellow |
| 12 | 102.2 | 4 | 15.9 | Golden yellow |

All sample solutions appeared as a clear oil (single phase) and had a moderate viscosity. The reason for the colour differences is unknown.

These results show that the generation of delta-9 THC from cannabidiol did not occur when the esters of octanoic acid, namely, methyl octanoate and ethyl octanoate were used and POPG-Na and octanoic acid were omitted. In other words, omitting POPG-Na and octanoic acid from the formulations and replacing the octanoic acid with esters of octanoic acid led to lower to nonexistent generation of delta-9 THC from cannabidiol. Furthermore, these results show that terminal sterilization did not compromise the chemical and physical stability of the formulations.

### Additional Embodiments

The following additional embodiments are in accordance with the invention.

TABLE 12

| | 13 | 14 | 15 | 16 | 17 | 18 | 19 | 20 |
|---|---|---|---|---|---|---|---|---|
| Ingredient | % w/v | | | | | | | |
| cannabidiol | 10 | 10 | 15 | 15 | 20 | 20 | 30 | 30 |
| Miglyol | 18 | 17 | 16 | 15 | 14 | 13 | 12 | 11 |
| 812N | | | | | | | | |
| Benzyl | 1 | 0.8 | 1.2 | 1.3 | — | — | 0.9 | 1.1 |
| alcohol | | | | | | | | |
| Methyl | QS | — | QS | — | QS | — | QS | — |
| octanoate | | | | | | | | |
| Ethyl | — | QS | — | QS | — | QS | — | QS |
| octanoate | | | | | | | | |

### LIST OF ITEMS

The following is a non-exhaustive list of items provided by the invention.

Item 1. An injectable cannabidiol formulation for non-IV parenteral administration comprising, consisting essentially of, or consisting of:
i. a solvent system comprising (i) an effective amount of at least one triglyceride oil; and (ii) an effective amount of at least one C1-C3 alkyl ester of a C6-C22 fatty acid; and
ii. an effective amount of cannabidiol; wherein the formulation is free of water and ethanol, has a viscosity less than about 35 cP, and is phase stable and chemically stable.

Item 2. The formulation of item 1, wherein the at least one C1-C3 alkyl ester of a C6-C22 fatty acid is selected from the group consisting of methyl octanoate, ethyl octanoate, isopropyl myristate, ethyl caprate, methyl oleate, ethyl oleate, and combinations thereof.

Item 3. The formulation of item 2, wherein the at least one C1-C3 alkyl ester of C6-C22 fatty acid is selected from the group consisting of methyl octanoate, ethyl octanoate, and combinations thereof.

Item 4. The formulation of any one of items 1 to 3, wherein the at least one triglyceride oil is at least one medium chain triglyceride.

Item 5. The formulation of item 4, wherein the at least one medium chain triglyceride comprises esters of fatty acids in the following amounts: 0 to 2% w/w ester of a C6 fatty acid, 50 to 65% w/w ester of a C8 fatty acid, 30 to 45% w/w ester of a C10 fatty acid, <2% w/w ester of a C12 fatty acid, <1% w/w ester of a C14 fatty acid, and <1% w/w ester of C16 or longer chain fatty acids.

Item 6. The formulation of any one of items 1 to 5, wherein the amount of the at least one triglyceride oil is at least about 10% w/v.

Item 7. The formulation of any one of items 1 to 6, wherein the amount of the at least one triglyceride oil is up to about 50% w/v.

Item 8. The formulation of any one of items 1 to 7, wherein the amount of the at least one C1-C3 alkyl ester of a C6 to C22 fatty acid is at least about 25% w/v.

Item 9. The formulation of any one of items 1 to 8, wherein the amount of the at least one C1-C3 alkyl ester of a C6 to C22 fatty acid is up to about 65% w/v.

Item 10. The formulation of any one of items 1 to 9, wherein the ratio of the (i) at least one triglyceride oil to the (ii) at least one C1 to C3 alkyl ester of a C6 to C22 fatty acid is from about 2:1 to about 1:7, or from about 1:1 to about 1:7, or from about 2:1 to about 1:6, or from about 1:1 to about 1:6.

Item 11. The formulation of any one of items 1 to 10, wherein the cannabidiol has a purity of at least 98.5, 99, 99.5, or 99.8%.

Item 12. The formulation of any one of items 1 to 11, wherein the cannabidiol is present in an amount of at least about 100 mg/mL and up to about 150 mg/mL, 200 mg/ml, 250 mg/mL, 300 mg/mL, or 350 mg/mL.

Item 13. The formulation of any one of items 1 to 12, wherein the formulation has a viscosity of less than about 30 cP, about 25 cP, or about 20 cP.

Item 14. The formulation of any one of items 1 to 13, further comprising, consisting essentially of, or consisting of at least one additional pharmaceutically acceptable excipient.

Item 15. The formulation of item 14, wherein said at least one additional pharmaceutically acceptable excipient is selected from the group consisting of additional lipophilic solvents, preservatives, antioxidants, anesthetic compounds, viscosity modifying agents, and pK modifying agents.

Item 16. The formulation of item 14 or 15, wherein benzyl alcohol is present in an amount from about 0.5% w/v to about 1.5% w/v.

Item 17. The formulation of any one of items 14 to 16, wherein an effective amount of at least one lipophilic antioxidant is present and is selected from the group consisting of propyl gallate, Vitamin E, ascorbyl palmitate, butylated hydroxytoluene (BHT) and butylated hydroxyanisole (BHA).

Item 18. The formulation of any one of items 1 to 17, wherein the solvent system consists of (i) an effective amount of at least one triglyceride oil; and (ii) an effective amount of at least one C1-C3 alkyl ester of a C6-C22 fatty acid; and (optionally), an effective amount of benzyl alcohol.

Item 19. The formulation of item 18, wherein the solvent system consists of (i) an effective amount of at least one medium chain triglyceride oil; and (ii) an effective amount of at least one of methyl octanoate and ethyl octanoate; and (optionally), an effective amount of benzyl alcohol.

Item 20. The formulation of any one of items 1 to 19, wherein the formulation is free of POPG-Na.

Item 21. The formulation of any one of items 1 to 19, wherein the formulation is free of phosphatidylglycerols and/or salts thereof.

Item 22. The formulation of any one of items 1 to 21, wherein the formulation is free of octanoic acid.

Item 23. The formulation of any one of items 1 to 21, wherein the formulation is free of C6-C22 fatty acids.

Item 24. The formulation of any one of items 1 to 21, wherein the formulation is free of fatty acids.

Item 25. A kit comprising (i) a container which comprises the formulation of any one of items 1 to 24; and (ii) instructions for use of the formulation.

Item 26. The kit of item 25, wherein the container is part of a single-dose or multi-dose injector, vial, cartridge, or pre-filled syringe.

The embodiments described above are by way of example only and are not intended to limit the scope of the invention as described herein and defined by the following claims.

1. An injectable cannabidiol formulation for non-IV parenteral administration comprising:
a. a solvent system comprising (i) an effective amount of at least one triglyceride oil; and (ii) an effective amount of at least one C1-C3 alkyl ester of a C6-C22 fatty acid;
b. an effective amount of cannabidiol; and
c. at least one antioxidant;
wherein the formulation is free of water and ethanol, has a viscosity less than about 35 cP, and is phase stable and chemically stable.

2. The formulation of claim 1, wherein the at least one C1-C3 alkyl ester of a C6-C22 fatty acid is selected from the group consisting of methyl octanoate, ethyl octanoate, isopropyl myristate, ethyl caprate, methyl oleate, and ethyl oleate.

3. The formulation of claim 2, wherein the at least one C1-C3 alkyl ester of C6-C22 fatty acid is selected from the group consisting of methyl octanoate and ethyl octanoate.

4. The formulation of claim 1, wherein the at least one triglyceride oil is at least one medium chain triglyceride.

5. The formulation of claim 4, wherein the at least one medium chain triglyceride comprises esters of fatty acids in the following amounts: 0 to 2% w/w ester of a C6 fatty acid, 50 to 65% w/w ester of a C8 fatty acid, 30 to 45% w/w ester of a C10 fatty acid, <2% w/w ester of a C12 fatty acid, <1% w/w ester of a C14 fatty acid, and <1% w/w ester of C16 or longer chain fatty acids.

6. The formulation of claim 1, wherein the amount of the at least one triglyceride oil is at least about 10% w/v.

7. The formulation of claim 1, wherein the amount of the at least one triglyceride oil is up to about 50% w/v.

8. The formulation of claim 1, wherein the amount of the at least one C1-C3 alkyl ester of a C6 to C22 fatty acid is at least about 25% w/v.

9. The formulation of claim 1, wherein the amount of the at least one C1-C3 alkyl ester of a C6 to C22 fatty acid is up to about 65% w/v.

10. The formulation of claim 1, wherein the ratio of the (i) at least one triglyceride oil to the (ii) at least one C1 to C3 alkyl ester of a C6 to C22 fatty acid is from about 2:1 to about 1:7, or from about 1:1 to about 1:7, or from about 2:1 to about 1:6, or from about 1:1 to about 1:6.

11. The formulation of claim 1, wherein the cannabidiol has a purity of at least 98.5, 99, 99.5, or 99.8%.

12. The formulation of claim 1, wherein the cannabidiol is present in an amount of at least about 100 mg/mL and up to about 150 mg/mL, 200 mg/ml, 250 mg/mL, 300 mg/mL, or 350 mg/mL.

13. The formulation of claim 1, wherein the formulation has a viscosity of less than about 30 cP, about 25 cP, or about 20 cP.

14. The formulation of claim 1, further comprising at least one additional pharmaceutically acceptable excipient.

15. The formulation of claim 14, wherein said at least one additional pharmaceutically acceptable excipient is selected from the group consisting of additional lipophilic solvents, preservatives, anesthetic compounds, viscosity modifying agents, and pK modifying agents.

16. The formulation of claim 1, wherein an effective amount of benzyl alcohol is present.

17. The formulation of claim 16, wherein the benzyl alcohol is present in an amount from about 0.5% w/v to about 1.5% w/v.

18. The formulation of claim 1, wherein the at least one antioxidant is selected from the group comprising propyl gallate, Vitamin E, ascorbyl palmitate, butylated hydroxytoluene (BHT) and butylated hydroxyanisole (BHA).

19. The formulation of claim 1, wherein the solvent system consists of (i) an effective amount of at least one triglyceride oil; and (ii) an effective amount of at least one C1-C3 alkyl ester of a C6-C22 fatty acid; and an effective amount of benzyl alcohol.

20. The formulation of claim 19, wherein the solvent system consists of (i) an effective amount of at least one medium chain triglyceride oil; and (ii) an effective amount of at least one of methyl octanoate and ethyl octanoate; and an effective amount of benzyl alcohol.

21.-27. (canceled)
